(19) United States
(12) Patent Application Publication
(10) Pub. No.: US 2011/0060280 A1
(43) Pub. Date: Mar. 10, 2011

(54) ADHESIVE SKIN PATCH WITH PUMP FOR SUBCUTANEOUS DRUG DELIVERY

(76) Inventors: Sean Caffey, Manhattan Beach, CA (US); Po-Ying Li, Chino Hills, CA (US); Yu-Chong Tai, Pasadena, CA (US)

(21) Appl. No.: 12/875,266

(22) Filed: Sep. 3, 2010

Related U.S. Application Data
(60) Provisional application No. 61/239,836, filed on Sep. 4, 2009.

(51) Int. Cl. A61M 5/168; A61M 35/00

(52) U.S. Cl. 604/151; 604/305

(57) ABSTRACT

In various embodiments, a drug-delivery device includes a skin patch with an integral delivery vehicle adherable to a patient's skin. An exterior surface of the patch defines an envelope within which is disposed a programmable drug pump including a reservoir, a cannula for conducting liquid from the reservoir to the delivery vehicle, and a mechanism for forcing liquid from the reservoir through the cannula and into the delivery vehicle.

### CROSS-REFERENCE TO RELATED APPLICATIONS

This application claims priority to and the benefit of, and incorporates herein by reference in its entirety, U.S. Provisional Patent Application No. 61/239,836, which was filed on Sep. 4, 2009.

### TECHNICAL FIELD

In various embodiments, the invention relates to pumps for delivering a drug, and in particular to pumps configurable as a skin patch.

### BACKGROUND

As patients live longer and are diagnosed with chronic and often debilitating ailments, the result will be an increased need for improvements to the speed, convenience, and efficacy of drug delivery. For example, many chronic conditions, including multiple sclerosis, diabetes, osteoporosis, and Alzheimer's disease, are incurable and difficult to treat with currently available therapies: oral medications have systemic side effects; injections may require a medical visit, can be painful, and risk infection; and sustained-release implants must typically be removed after their supply is exhausted (and offer limited ability to change the dose in response to the clinical picture). In recent decades, several types of portable drug delivery devices have been developed, including battery-powered mini pumps, implantable drug dispensers, and diffusion-mediated skin patches.

Drug-delivery devices configured as adhesive skin patches provide several advantages over competing delivery technologies for the treatment of chronic diseases. They are compact, disposable, and incur relatively low manufacturing costs. Relative to other drug-delivery options, they are non-invasive since they require the simple adhesion to the skin of a patch-type device containing a reservoir that stores a drug or therapeutic agent. This type of device also provides flexibility in terms of where it can be applied, since the skin serves as a large accessible surface for the patch device. In several existing applications, patch-based devices rely on transdermal absorption for drug delivery, e.g., diffusion of the drug across the skin. However, because the skin exhibits low permeability and functions as a barrier to prevent molecular transport of foreign agents into the body, effective diffusion-based drug penetration is generally limited to drugs with low molecular weights. Accordingly, transdermal drug delivery is typically compatible with only a limited number of pharmaceutical agents and suitable only for the handful of diseases they treat. Another limitation of transdermal skin patches is that penetration across the contact area can often be heterogeneous and uncontrolled. Treatments for a number of chronic diseases currently require the administration of a drug or therapeutic agent either continuously or at specific times or time intervals in high controlled doses.

Several chronic diseases are currently treatable only with drugs that require subcutaneous drug delivery. Subcutaneous injections take advantage of the lack of blood flow to the subcutaneous layer, which allows the administered drug to be absorbed more slowly over a longer period of time. However, these types of injections typically must be administered either by the patient or a medical practitioner anywhere from several times a day to once every few weeks. Frequent injections can result in discomfort, pain, and inconvenience to the patient. Self-administration also leaves open the possibility for non-compliance or errors in dosage events.

There is a need, therefore, for a skin patch-based delivery system capable of delivering highly controlled dosages of drug at regular intervals or intermittently, depending on the needs of the patient.

### SUMMARY OF THE INVENTION

In general, in one aspect, embodiments of the invention feature a drug-delivery device that includes a patch adherable to a patient's skin. An exterior surface of the patch defines an envelope within which are disposed at least one programmable drug pump including a reservoir, a cannula for conducting liquid from the reservoir to a delivery vehicle integrated with the patch, and a mechanism for forcing liquid from the reservoir through the cannula and into the delivery vehicle. All of these components are integral with the patch. A sensor associated with the cannula monitors a parameter of a fluid within the cannula and feedback circuitry, responsive to the sensor, adjusts operation of the drug pump.

In one embodiment, the delivery vehicle is a sponge positioned for contact with the skin with the patch affixed thereto. In an alternative embodiment, the delivery vehicle is a lancet insertable into the skin with the patch affixed thereto. The lancet may be retractable or wirelessly actuable. In an alternative embodiment, the cannula and catheter can be separated from the body of the pump while using an external needle lancet system to drive the catheter into the skin. In various embodiments, the pump may be electrolytically driven and the reservoir may be refillable.

In some embodiments, the patch includes first and second opposed surfaces, where the first surface is adherable to the skin and the second surface is under a hydrophobic layer to retain moisture within the patch. The patch may also be flexible, and the sensor may be one or more of a flow sensor, a pressure sensor, or a thermal sensor.

In general, in another aspect, embodiments of the invention feature a drug-delivery device including a patch adherable to a patient's skin and a plurality of drug pumps integral with the patch and residing within an envelope defined by the patch. Some embodiments feature a common reservoir and at least one cannula for conducting liquid therefrom to at least one delivery vehicle in fluid communication with the drug pumps, so that the pumps may force liquid from the common reservoir through the cannula(s) and into the delivery vehicle(s). A controller for selectively activating the pumps to achieve a programmed dosage may also be included. In other embodiments, multiple reservoirs allow for two or more drugs to be delivered at different intervals using the same or separate cannulas.

In one embodiment, each of the pumps fluidly communicates with a separate delivery vehicle (forming, for example, an array of microneedles that results in less perceived pain by the patient). In an alternative embodiment, each of the pumps fluidly communicates with a common delivery vehicle. The drug-delivery device may also include a sensor associated with each at least one cannula for monitoring a parameter of a fluid therein and feedback circuitry, responsive to the at least one sensor, for adjusting operation of the drug pumps.

In general, in yet another aspect, embodiments of the invention feature a drug-delivery device including a patch adherable to a patient's skin and, integral with the patch and residing within an envelope defined by the patch, at least one programmable drug pump including a reservoir, a cannula for conducting liquid from the reservoir to a delivery vehicle integrated with the patch, and a mechanism for forcing liquid from the reservoir through the cannula and into the delivery vehicle. The drug-delivery device may also include a flexible bladder downstream of the reservoir and upstream of an outlet of the cannula for receiving fluid from the reservoir and discharging it into the cannula. This has the advantage of saving power, since the power-hungry electrolysis system is active just long enough to pump fluid from the drug reservoir into the flexible bladder reservoir; the bladder compresses the drug out the catheter (a check valve is used to prevent backflow into the reservoir) even while the electrolysis is turned off.

In various embodiments, the drug-delivery device may also include a check valve between the reservoir and the flexible bladder, a sensor associated with the flexible bladder, and feedback circuitry, responsive to the sensor, for adjusting operation of the drug pump. The sensor may detect depletion of the flexible bladder and the feedback circuitry may cause the drug pump to operate so as to fill the flexible bladder.

In general, in another aspect, the invention features a drug-delivery device including a patch adherable to a patient's skin, and, integral with the patch and residing within an envelope defined by the patch, a lancet wirelessly actuable for insertion into a patient's skin in contact with the patch. The device also includes at least one programmable drug pump including a reservoir, a cannula for conducting liquid from the reservoir to the lancet, and a mechanism for forcing liquid from the reservoir through the cannula and into a delivery vehicle.

These and other objects, along with advantages and features of the embodiments of the present invention herein disclosed, will become more apparent through reference to the following description, the accompanying drawings, and the claims. Furthermore, it is to be understood that the features of the various embodiments described herein are not mutually exclusive and can exist in various combinations and permutations, even if not made explicit herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 schematically illustrates, in bottom view, a drug-delivery device in accordance with one embodiment of the invention;

FIGS. 2A and 2B schematically illustrate, in isometric views, a drug-delivery used in accordance with one embodiment of the invention;

FIG. 2C schematically illustrates, in schematic elevational cross-section, a delivery mechanism for use with various embodiments of the invention;

FIG. 3 schematically illustrates, in elevational cross-section, an electrolysis pump for use with the device illustrated in FIG. 1;

FIG. 4 schematically illustrates, in a block diagram, the configuration of a drug-delivery device in accordance with one embodiment of the invention;

FIGS. 5A and 5B schematically illustrate, in cut-away isometric views, a drug-delivery device in accordance with an alternative embodiment of the invention;

FIGS. 6A-6C schematically illustrate, in top view, drug-delivery devices with multiple pumps in accordance with other embodiments of the invention; and

FIG. 7 schematically illustrates, in bottom view, a drug-delivery device with a flexible downstream bladder in accordance with yet another embodiment of the invention.

### DESCRIPTION

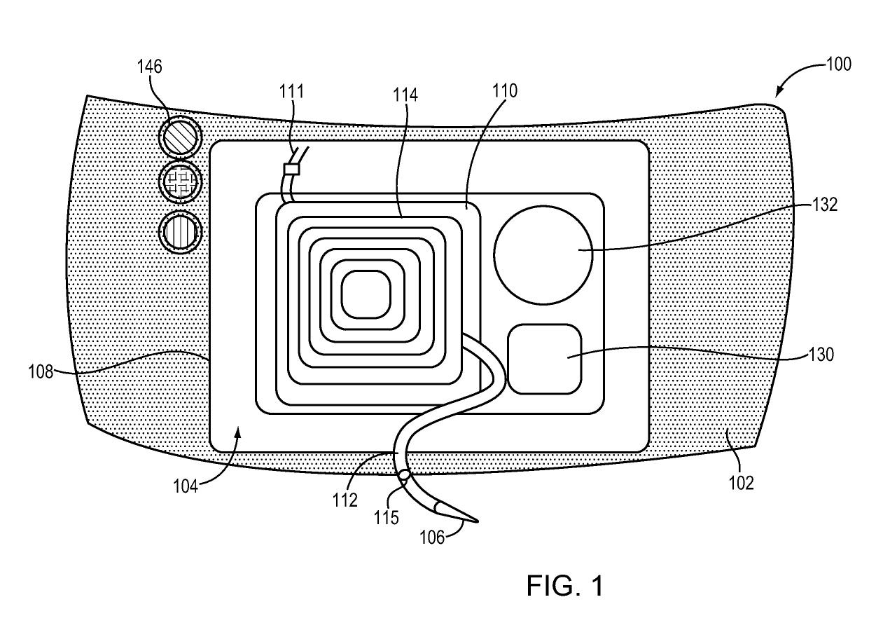

In general, embodiments of the present invention pertain to patches adherable to the skin of a patient with integral drug-delivery pumps, and may be employed in connection with various types of skin patches. Refer first to FIG. 1, which illustrates an embodiment 100 of a drug-delivery device in accordance with the invention. The drug-delivery device 100 includes an adhesive patch 102 (e.g., an adhesive bandage) and, affixed to a bottom surface thereof, a programmable drug pump assembly 104. A delivery vehicle 106 extends from the pump assembly 104 to facilitate transfer of drug from the pump to the wearer. A clear portion (not shown) of the adhesive patch 102 may be provided about the delivery vehicle 106 so a patient can confirm that the delivery vehicle 106 did not pierce a vein when applied to the skin, as evidenced by a lack of hematoma or blood bruising visible through the window.

The adhesive patch 102 is generally fabricated from a flexible material that conforms to the contours of the patient's skin and attaches via an adhesive on the illustrated backside surface that contacts a patient's skin. The adhesive may be any material suitable and safe for application to and removal from human skin Many versions of such adhesives are known in the art, though utilizing an adhesive with gel-like properties may afford a patient particularly advantageous comfort and flexibility. The adhesive may be covered with a removable layer to preclude premature adhesion prior to the intended application. As with commonly available bandages, the removable layer should not reduce the adhesion properties of the adhesive when removed.

On the bottom surface of the patch 102, the various components of the drug pump assembly 104 are held within a housing 108 that is either fully self-contained or, if defined as discrete, intercommunicating modules, reside within a spatial envelope that is wholly within (i.e., which does not extend beyond in any direction) the perimeter of the patch 102. For example, the housing 108 may be fully sealed and watertight except for where the delivery vehicle 112 extends from the patch 102. The housing 108 protects the components of the drug pump assembly 104 and prevents the unintentional disassembly of the drug-delivery device 100.

In one embodiment, where the patch 102 is made from a flexible material, the portion of the upper surface opposite the housing 108 may be constructed from or capped with an inflexible material. The inflexible material may effectively form a shell to protect the drug pump assembly 104 and prevent disruption of its operation from a number of causes, such as changes in the external environment (e.g., pressure) and accidental contact.

Alternatively or in addition, the upper surface of the patch 102 may have thereon (or may consist of) a layer made of silicone rubber, glass, or a hydrophobic coating to retain moisture within the patch 102. Covering the drug pump assembly 104 with a protective material, such as silicone or epoxy, also protects the pump components. The protective material may be applied to the flexible material of the patch 102 to adhere thereto, sandwiching the housing 108 therebetween. Adhesion between the protective and flexible materials may be achieved with any of a number of known manufacturing steps for combining materials, such as applying epoxy to the materials or heat-sealing the materials together.

The delivery vehicle 106 may be any device suitable for delivering a fluid to a patient. In various embodiments, the delivery vehicle 106 is configured to deliver fluid to the skin surface for absorption (e.g., via a sponge) or to deliver fluid to the subcutaneous layer directly (e.g., via a lancet). For direct subcutaneous delivery applications, the delivery vehicle 106 must be of sufficient strength and flexibility to penetrate the subcutaneous layer without breaking or bending. Examples of such materials include, but are not limited to, stainless steel, silicon, polyurethane, and various composite materials as are well-known in the art.

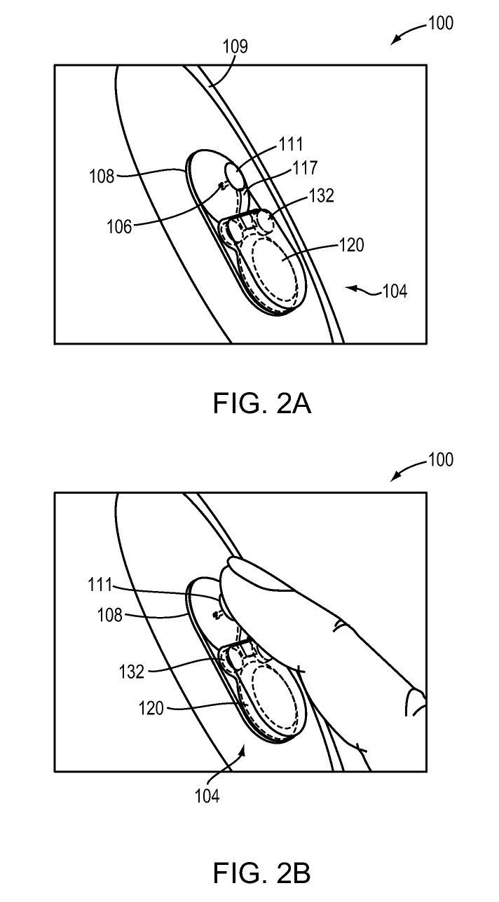

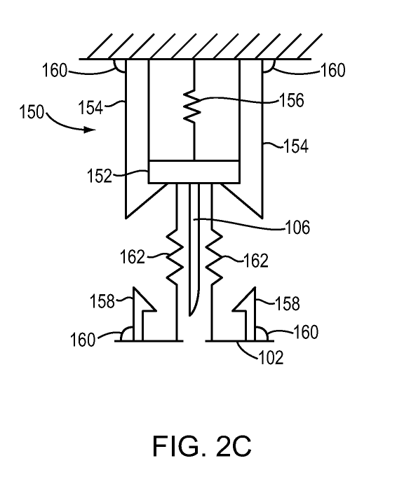

The delivery vehicle 106 may be manually forced to or through the surface of the skin, as depicted in FIGS. 2A and 2B, depending on the application. In certain embodiments, the delivery vehicle 106 is a delivery vehicle biased away from the skin 109 and driven into the skin against the bias. The delivery vehicle 106 may be actuated by a manual trigger, such as a button 111. Pressing the button 111 drives the delivery vehicle 106 into the skin and also activates the pump electronics (described below), e.g., by bringing electrical contacts together. The button 111 may be hinged by, for example, a living hinge 117 that biases it in the retracted position. When the button 111 is pressed, overcoming the hinge bias, a catch holds it in place (and the delivery vehicle 106 in position) until the button 111 is pressed again. In addition to manual release by means of a second depression of the button 111, the catch may be electromagnetically configured for release in response to a signal from the pump circuitry (after a predetermined amount of drug is sensed to have been delivered to the wearer) or from a wireless device.

A suitable mechanism 150 facilitating retractable insertion of the delivery vehicle 106 through the skin is depicted in FIG. 2C. The mechanism 150 may operate mechanically or electromechanically. In the illustrated configuration, the delivery vehicle 106 is a lancet coupled to a lancet support 152 held in a retracted position by a pair of first catch elements 154 against a first biasing elastic element 156, such as a spring or a sponge. The lancet 106 is actuated (or released), either manually or in response to a signal from the pump or a wireless device, by briefly opening the first catch elements 154, and also a pair of second catch elements 158, about associated hinges 160. The first elastic element 152 quickly forces the lancet 106 into the skin, where the lancet support 152 is restrained by the second catch elements 158. Additional second elastic elements 162, biasing the lancet 106 toward the patch 102, may be included to retract the lancet 106 at a desired time, such as following the administration of a full dose. The lancet 106 may be actuated for retraction either manually or, once again, by means of a signal (received from a wireless source or from the pump, e.g., when a full dose has been dispensed) by briefly opening the second catch elements 158 and the first catch elements 154 about hinges 160. The second elastic elements 162 quickly force the lancet 106 back within the patch 102, where the lancet support 152 is again retained by the first catch elements 154. To facilitate automatic operation, the first and second catch elements 154, 158 may be mounted on a piezoelectric material, which undergoes strain upon application of voltage thereto, thus opening the first and second catch elements 154, 158. Removal of the voltage from the piezoelectric material relieves the strain, thereby restoring the first and second catch elements 154, 158 to a closed configuration.

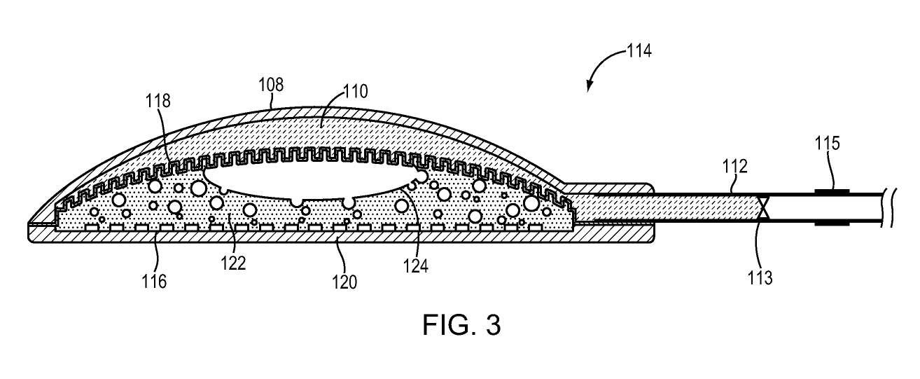

As shown in FIGS. 1 and 3, the drug pump assembly 104 may include a reservoir 110, a cannula 112, and a pump 114. The reservoir 110 is a chamber configured to store a drug in liquid form. The reservoir 110 may also include a refill port 111 to allow for the introduction of additional drug. In some embodiments, the reservoir 110 is capable of holding between approximately one and ten mL of a drug and has an active operational lifetime of, e.g., 30 minutes to 75 hours, though the capacity and operational lifetime of the reservoir 110 is easily adjusted by altering the size of the reservoir 110 and the rate at which the drug is administered. The cannula 112 is fluidically coupled to the reservoir 110 to provide a fluid path from the reservoir 110 to (and through) the delivery vehicle 106. The cannula 112 may contain a check valve 113 (see FIG. 3) to prevent blood or interstitial fluid from entering the reservoir 110 and spoiling the drug. The cannula 112 can be made of substantially impermeable tubing, such as medical-grade plastic.

The cannula 112 may include a sensor 115 for monitoring a parameter, such as flow rate, of a fluid within the cannula 112. In general, the sensor 115 may be a flow, thermal, time of flight, pressure, or other sensor, as are well-known in the art. In one embodiment, the sensors 115 are fabricated, at least in part, from parylene, which is a biocompatible, thin-film polymer. Advantageously, this enables the sensors 115 to be fully integrated into a parylene-based drug pump 100 (as described below). It may be desirable for parylene to be the only material in contact with the fluid flowing through the cannula 112 (e.g., to ensure biocompatibility and also to protect the other elements in the sensors 115).

A thermal flow sensor uses a resistive heater to locally heat the fluid flowing in proximity to the sensor 115. The temperature of the flowing fluid can then be measured using one or more miniature resistive temperature devices, providing an indication of the flow rate. A time-of-flight sensor generates a tracer pulse in the fluid flowing within the cannula 112, and then measures the time that it takes for this pulse to traverse a certain distance. This measured time is defined as the “time of flight” and corresponds to the linear fluid velocity, which may be translated into a volumetric flow rate. Multiple pressure sensors may be used to detect a difference in pressure and calculate the flow rate based on a known laminar relationship.

A pressure sensor located in or on the cannula 112, or within the reservoir 110 (e.g., at the outlet port leading to the cannula), can also be used to measure and monitor the local pressure. Pressure sensing can be used to warn of improper pump operation or as an indirect measure of flow rate. For example, if knowledge of the pressure in the delivery vehicle 106 is required during dosing, then the sensor 115 can be placed in either of two places: (i) inside the cannula 112 and at its distal tip, or (ii) outside the cannula 112 and at its distal tip. Advantageously, placement of the sensor 115 at the distal tip of the cannula 112 prevents flow-related pressure drops inside the cannula 112 from causing an error in the pressure reading.

The pump 114 forces liquid from the reservoir 110 through the cannula 112 and into the delivery vehicle 106. In various embodiments, the pump 114 is an electrolytic pump, as depicted in FIG. 3. A suitable electrolytic pump 114 includes an electrolysis chamber 116, one surface of which is defined by a diaphragm 118. The reservoir 110 is located on one side of the electrolysis chamber 116 (and within the housing 108). The diaphragm 118 defines the lower boundary of the reservoir 110 as well as the upper boundary of the electrolysis chamber 116. A portion of the outer surface of the housing 108 defines the upper boundary of the reservoir 110. The diaphragm 118 may be molded out of parylene (or microfabricated). The electrolysis chamber 116 contains a series of electrolysis electrodes 120 and an electrolyte 122 in liquid form. In operation, when current is supplied to the electrolysis electrodes 120, the electrolyte 122 evolves gas 124, expanding the diaphragm 118 (i.e., moving the diaphragm 118 upwards in FIG. 3) and forcing liquid (e.g., drug) out of the drug reservoir 110, into and through the cannula 112, and out the distal end thereof to the delivery vehicle 106 (see FIG. 1). The diaphragm 118 may be corrugated or otherwise folded to permit a large degree of expansion without sacrificing volume within the drug reservoir 110 when the diaphragm 118 is relaxed. When the current is stopped, the electrolyte gas 124 condenses back into its liquid state 122, and the diaphragm 118 recovers its space-efficient corrugations. The electrolytic pump 114 may be smaller and more portable than other pumps because of its lack of rigidly moving parts. A high degree of pressure (i.e., greater than 20 psi) can be generated, allowing the drug pump assembly 104 to overcome any biofouling or blockages in the system.

The diaphragm 118 may be made with or from parylene polymer using microfabrication techniques. The electrodes 120 may be any suitable metal, such as platinum, titanium, gold, and copper, among others. Titanium has the advantage of not causing recombination of hydrogen and oxygen gas, making for a more efficient system compared to platinum, which causes hydrogen and oxygen gas to combine into water in its presence. It may be desirable, however, for some refillable devices to employ platinum electrodes.

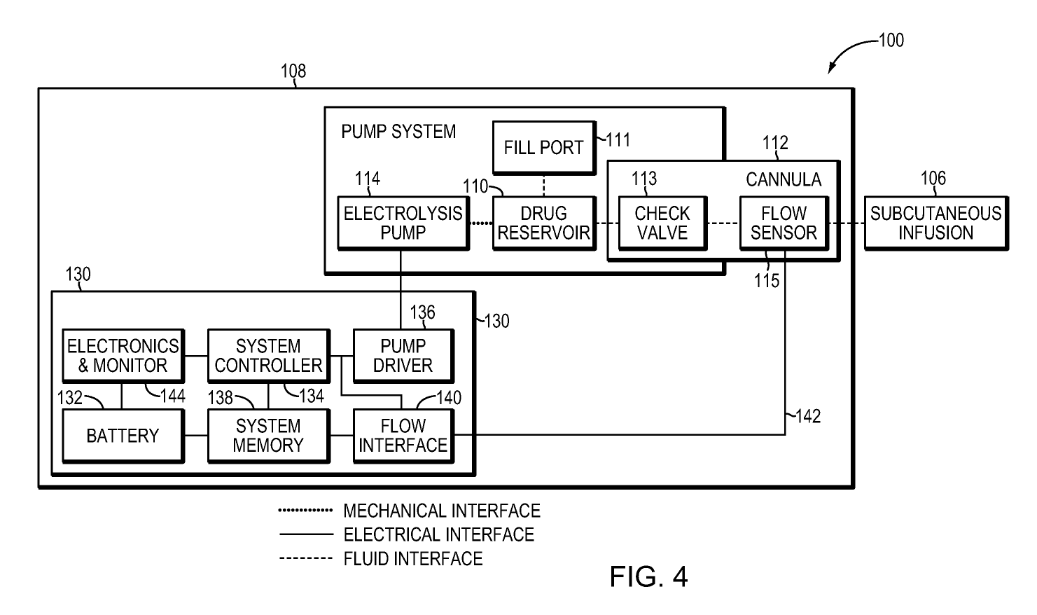

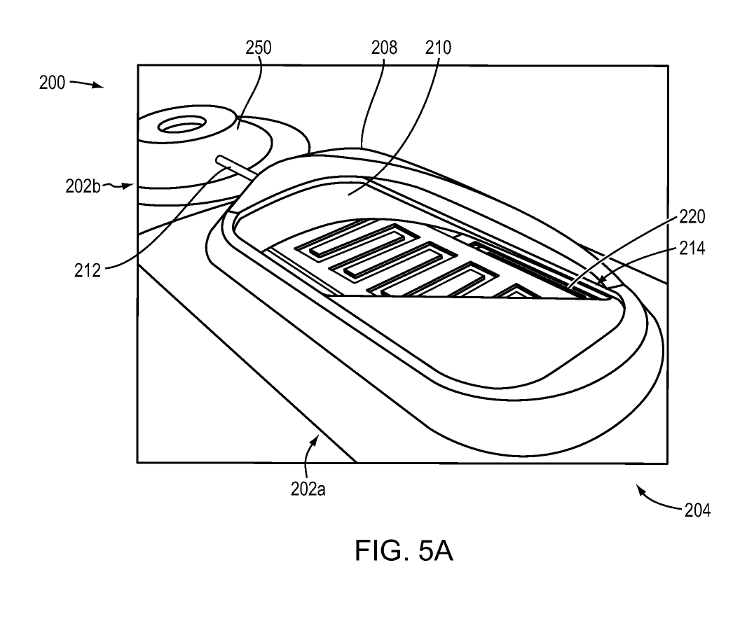

The drug-delivery device 100 also includes a control system 130, as depicted in FIG. 4. The illustrated control system 130 includes a battery 132 for powering the drug-delivery device 100, a programmable system controller 134 for controlling the drug-delivery device 100, a pump driver 136 for controlling the pump 114, a system memory 138, a flow interface 140 for relaying information obtained through feedback circuitry 142 from the sensor 114 to the system controller 134, and as appropriate to the application, other electronics and monitoring components generically indicated at 144. A multi-LED display 146 (see FIG. 1) may also be included to indicate the current status of the device 100. The components of system 130 may be mounted on a circuit board, which is desirably flexible and/or may be an integral part of the pump housing.

The system controller 134 receives signals from the flow sensor 115 and interprets these to measure the amount of liquid dispensed through the cannula 112. Executable instructions in the system memory 138, which are straightforwardly provided without undue experimentation, dictates the actions of the system controller 134 in general and in response to the received signals in particular. For example, the system controller may be programmed to dispense a particular amount of liquid at fixed intervals. As these intervals occur, the system controller 134 actuates the delivery vehicle 106 and then the electrolysis pump 114. When the signals from the flow sensor 115 indicate that the proper dosage has been administered, the system controller 134 terminates the operation of the pump 114 and, if appropriate, causes retraction of the delivery vehicle 106.

The system controller 134 also assesses the flow through the cannula 112 as reported by the flow sensor 115 and takes corrective action should the flow rate deviate sufficiently from a programmed or expected rate. For example, where the system controller 134 determines that a higher flow rate of drug is needed, it may increase the current to the electrolysis electrodes 120 to evolve greater gas in the electrolysis chamber 116, thereby more rapidly expanding the diaphragm 118 and increasing the fluid flow rate through the cannula 112. Alternatively, where the system controller 134 determines that a lower flow rate of drug is needed, it may decrease the current to the electrolysis electrodes 120 to evolve less gas in the electrolysis chamber 116, thereby reducing the rate of expansion of the diaphragm 118 and decreasing the fluid flow rate through the cannula 112. Depending upon the particular application for which the drug-delivery device 100 is employed, the flow rate requirements for fluid flowing through the cannula 112 may range from the nL/min to the μL/min flow scales.

The control system 130 is capable of controlling the drug-delivery device 100 to deliver either continuous infusion or intermittent drug delivery to the subcutaneous layer. For example, the stored instructions may implement a “dinner pump” where a 150 μL dose of insulin is needed immediately after dinner, but another 850 μL is dispensed at a “basal rate” over 6 hours while the patient sleeps. The drug-delivery device 100 may be configured to achieve sustained drug release over periods ranging from several hours to several months. The dosage events may be programmed to occur at specific times or time intervals, or they may take place in response to changing conditions in the patient. For example, in some embodiments, electronics 144 includes a conventional microelectronic communication module facilitating bidirectional wireless data transfer with an external transceiver, allowing a clinician to alter the programming in system memory 138 should the patient's condition change.

In one embodiment, the drug-delivery device 100 is automatically activated once the skin patch 102 is unwrapped and moisture is sensed. Other embodiments of the drug-delivery device 100 may be manually activated as described above. In some of these embodiments, for example, the pump 114 can be toggled on and off with a manual push. Optionally, the pump 114 can also be manually forced to speed up or slow down by means of wirelessly transmitted commands or manual control of user-accessible controls. In alternative embodiments, the pump 114 is activated when the lancet 106 is inserted into the skin. The device 100 may alert the patient that drug delivery is complete by, for example, issuing a signal or retracting the lancet 106, as previously discussed.

The battery 132 may be a non-rechargeable lithium battery approximating the size of batteries used in wristwatches, though rechargeable Li—PON, lithium polymer batteries, nickel-metal-hydride, and nickel cadmium batteries may also be used. Other devices for powering the drug-delivery unit 100, such as a solar cell or motion-generated energy system, may be used either in place of the battery 132 or supplementing a smaller battery. This can be useful in cases where the patient needs to keep the drug-delivery device 100 on for several days or more.

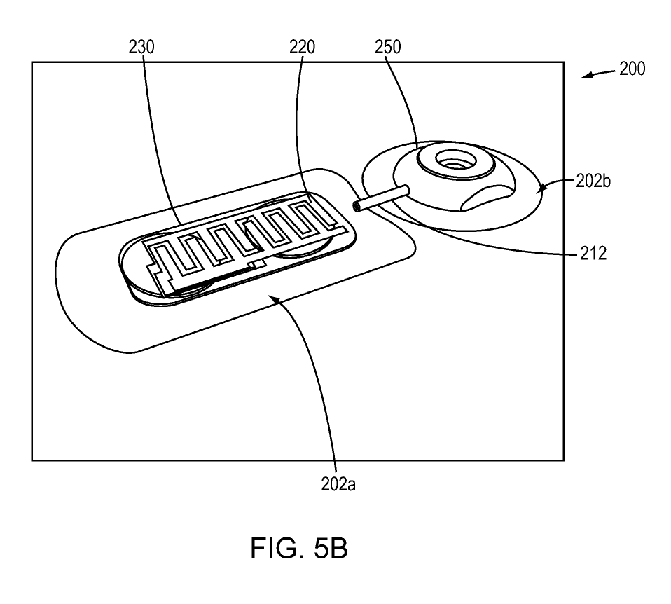

In another embodiment, as depicted in FIGS. 5A and 5B, a drug-delivery device 200 includes the same components as the drug-delivery device 100, but in a different configuration. The drug-delivery device 200 includes an adhesive patch in two parts, a drug pump portion 202a and a removable, replaceable infusion set portion 202b; FIG. 5B shows the device with the shell or case removed from the portion 202a. That portion includes a drug pump 204, a reservoir 210, a cannula 212, and an electrolytic pump 214 to move fluid from the reservoir 210 to the cannula 212 into a delivery vehicle which is part of an infusion set 250 on device portion 202b. A control system 230 is disposed below electrodes 220. The infusion set portion 202b includes the infusion set 250 and a fluid coupling for removably but sealably receiving the cannula 212. The infusion set 250 also includes a delivery vehicle and any of the mechanisms that may be associated with it, as discussed above in relation to the delivery vehicle 106. Both parts of the patch 202a, 202b each reside within a small, planar envelope, and each overlies a discrete adhesive patch 208. All operations of the drug-delivery device 200 may be identical to that of the drug-delivery device 100, as previously described. An advantage to the device 200 is the ability to leave the pump portion 202a in place while changing the infusion set 250, merely by manually disengaging the device portion 202b from the cannula 212 and lifting the portion 202b (and its adhesive patch) from the skin.

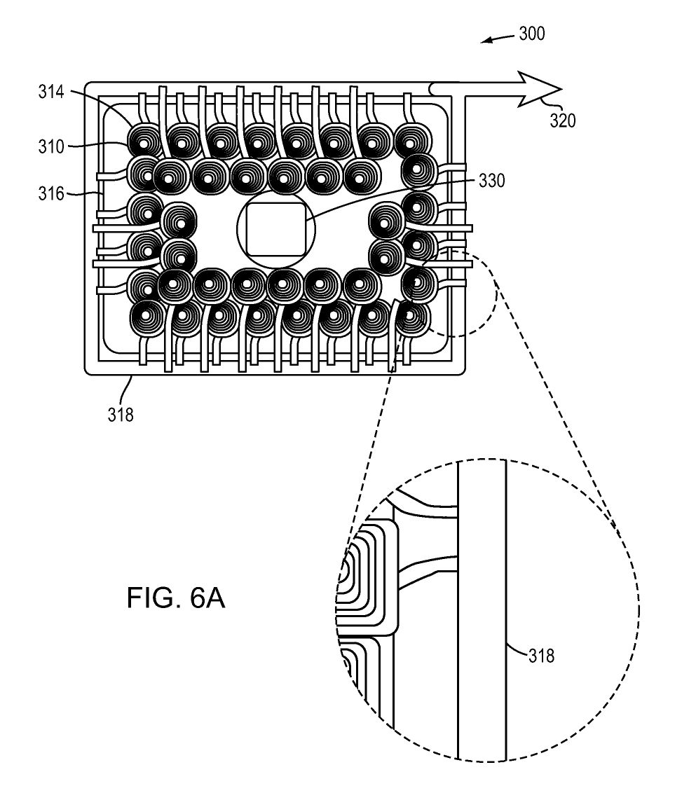

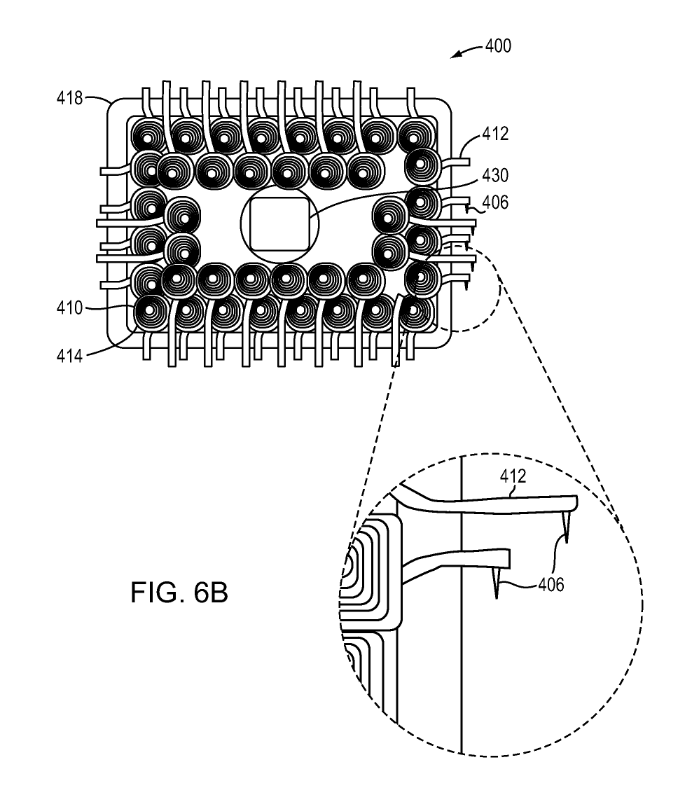

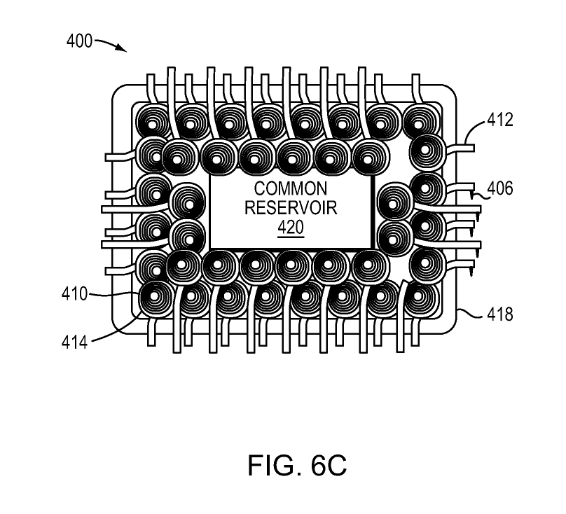

Some embodiments, as illustrated in FIGS. 6A-6C, contain multiple pumps on a single patch. Various configurations are possible: each pump with its own reservoir but sharing a delivery with one or more (or all) other pumps; each pump with its own reservoir and delivery vehicle; and a common reservoir accessed by all pumps, which may use one or more shared delivery vehicles or may each have its own delivery vehicle. With reference to FIG. 6A, a drug-delivery device 300 contains a plurality of reservoirs 310 and pumps 314 (each with the components shown in FIG. 1, but controlled by a single pump controller) that reside on a single adhesive patch 316. The patch 316 may have a sandwich configuration retaining a sponge or pad impregnated with saline solution (i.e., approximately 0.9% saline) for osmotic control. This may augment the flexibility of the patch 316 while also protecting the pumps 314 from mechanical damage and discouraging evaporation of drug. Each of the reservoirs 310 and the pumps 314 empty into a single conduit 318, which is in turn connected to a single cannula and delivery vehicle as indicated at 320. A control system 330 coordinates the operation of the pumps 314 in the manner described above. The volume of drug stored in each pump 314 may be the same or varied, and may be as little as 50 μL or less. The pumps 314 are arranged in an array and can function either independently or collectively to deliver variable dosage volumes, essentially achieving controllable dosage resolution equal to an average dosage delivered by each pump 314. The pumps 314 can be arrayed adjacent to each other on the same surface or stacked on top of one another (or both). In any arrangement, all of the pumps 314 and the reservoirs 310 remain within an envelope within the borders of the patch 316.

The reservoirs 310, each actuated by one or more individual pumps 314, can store different drugs, facilitating variable drug mixing through selective pump activation. Different drugs can be administered together as part of a drug “cocktail” or separately at different times, depending on the treatment regimen. These multiple reservoirs 310 may also facilitate mixing of agents, such as in the case where a first reservoir stores a first agent and a second reservoir stores a second agent. The first agent may be a drug that is stored in a “dormant” state with a half-life of several months, and the second agent may be a catalyst required for activating the first agent. By controlling the amount of the second agent that reacts with the first agent, the drug-delivery device 300 is able to regulate the potency of the delivered dosage. As noted, the drug-delivery device 300 may be programmed to deliver different drugs at different times, depending on the treatment regimen, and as explained above, in some embodiments pump operation can be altered through commands issued wirelessly to the pump. The array of pumps 314 can be broken into subsets, each of which administers a specific drug at an appropriate time.

In another embodiment, the drug-delivery device 300 includes only a single reservoir. The array of pumps 314 draw on the single reservoir to provide highly variable flow rates. If a very high flow rate is desired, all of the pumps 314 can simultaneously active. This allows fine, modular control over the overall flow rate, as well as potentially providing redundancy should any of the pumps fail.

FIGS. 6B and 6C depict another embodiment 400 in which each pump 414 has its own cannula 412 and delivery vehicle 406 on a single adhesive-backed patch 418. Each pump 414 may also be coupled to its own reservoir 410 (as shown in FIG. 6B), or all of the pumps 414 may share a common reservoir 420 (as shown in FIG. 6C). The multiple-outlet arrangement can provide uniform dosing throughout a contact area of the delivery vehicles 406. Parallel operation of the pumps 414 may lead to faster response times and better dosage control. This arrangement also improves the safety and efficacy of patch-based drug delivery by including redundant components that are capable of functioning independently. This prevents the failure of a single pump 414 from interrupting the operation of the drug-delivery device 400. Side effects, such as scarring and damage to the subcutaneous tissue layer, that result from frequently administered injections may be reduced or avoided, thereby improving quality of life for the patient. Administering several smaller doses over a larger surface area using multiple delivery vehicles 406 may also help to reduce systemic side effects occurring due to a high concentration of drug being delivered to a small target area.

In each of the drug-delivery devices 300, 400, other types of drug pumps 314, 414 may be used instead of the described electrolytic pumps, particularly those that rely on electro-osmotically actuated, pressure-driven, or mechanically driven mechanisms. Additionally, the pump microarrays may be microfabricated using MEMS processing. Titanium and steel are useful metals in this process.

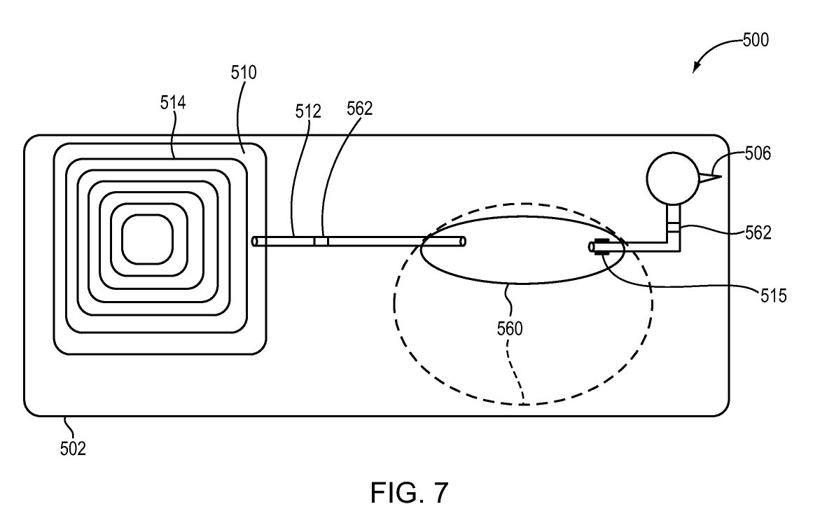

FIG. 7 depicts another embodiment of a drug-delivery device 500 with components identical to those of the drug-delivery device 100, including a housing 502 and a pump 514, with the addition of a flexible bladder 560 and a pair of check valves 562. The flexible bladder 560 may be made of an elastic polymer such as parylene, and is typically disposed between a reservoir 510 and a delivery vehicle 506 to serve as a variable-volume, intermediate storage reservoir. This allows a pump 514 to operate for a shorter duration (e.g., ten minutes) in order to fill the flexible bladder 560. Once the flexible bladder 560 is sufficiently full, the pump 514 can shut down and allow the flexible bladder 560 to force drug to the delivery vehicle 506 (either a single lancet or an array of machined needles) for an extended period of time (e.g., 50 minutes). In this manner, the drug-delivery device 500 can provide a constant flow rate without constant power. The check valves 562 may be disposed in a cannula 512 between the reservoir 510 and the flexible bladder 560 to prevent backflow, and in the cannula 512 between the flexible bladder 560 and the delivery vehicle 506 to prevent blood or interstitial fluid from entering the reservoir 510 and spoiling the drug. A sensor 515, such as a pressure sensor, may be disposed in the cannula 512 or the flexible bladder 560 to communicate to the pump control system when the pump 514 needs to restart to fill the flexible bladder 560. The sensor 515 may be of the types previously described, though using a pressure sensor can increase the consistency of the flow rate improving regulation of the filling cycles of the pump 514.

Having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A drug-delivery device comprising:
a patch adherable to a patient's skin;
integral with the patch and residing within an envelope defined entirely by an exterior surface of the patch, at least one programmable drug pump comprising (i) a reservoir, (ii) a cannula for conducting liquid from the reservoir to a delivery vehicle integrated with the patch, and (iii) a mechanism for forcing liquid from the reservoir through the cannula and into the delivery vehicle;
a sensor for monitoring a parameter of a fluid in the drug pump; and
feedback circuitry, responsive to the sensor, for adjusting operation of the drug pump.

2. The device of claim 1 wherein the sensor is associated with the cannula for monitoring flow therethrough.

3. The device of claim 1 wherein the sensor is a pressure sensor residing within the reservoir.

4. The device of claim 1 wherein the delivery vehicle is a lancet insertable into the skin with the patch affixed thereto.

5. The device of claim 4 wherein the lancet is retractable.

6. The device of claim 4 wherein the lancet is wirelessly actuable.

7. The device of claim 1 wherein the pump is electrolytically driven.

8. The device of claim 1 wherein the reservoir is refillable.

9. The device of claim 1 wherein the patch comprises first and second opposed surfaces, the first surface being adherable to the skin, and further comprising a hydrophobic layer over the second surface to retain moisture within the patch.

10. The device of claim 1 wherein the patch is flexible.

11. The device of claim 1 wherein the sensor is a flow sensor.

12. The device of claim 1 wherein the sensor is a pressure sensor.

13. The device of claim 1 wherein the sensor is a thermal sensor.

14. A drug-delivery device comprising:
a patch adherable to a patient's skin;
a plurality of drug pumps integral with the patch and residing within an envelope defined by the patch;
in fluid communication with the drug pumps, a common reservoir and at least one cannula for conducting liquid therefrom to at least one delivery vehicle integrated with the patch, the pumps forcing liquid from the common reservoir through the at least one cannula and into the at least one delivery vehicle; and
a controller for selectively activating the pumps to achieve a programmed dosage.

15. The device of claim 14 wherein each of the pumps fluidly communicates with a separate delivery vehicle.

16. The device of claim 14 wherein each of the pumps fluidly communicates with a common delivery vehicle.

17. The device of claim 14 further comprising:
a sensor associated with each said at least one cannula for monitoring a parameter of a fluid therein; and
feedback circuitry, responsive to the at least one sensor, for adjusting operation of the drug pumps.

18. A drug-delivery device comprising:
a patch adherable to a patient's skin;
integral with the patch and residing within an envelope defined by the patch, at least one programmable drug pump comprising (i) a reservoir, (ii) a cannula for conducting liquid from the reservoir to a delivery vehicle integrated with the patch, and (iii) a mechanism for forcing liquid from the reservoir through the cannula and into the delivery vehicle; and
a flexible bladder downstream of the reservoir and upstream of an outlet of the cannula, the bladder receiving fluid from the reservoir and discharging it into the cannula.

19. The device of claim 18 further comprising a check valve between the reservoir and the flexible bladder.

20. The device of claim 18 further comprising a sensor associated with the flexible bladder and feedback circuitry, responsive to the sensor, for adjusting operation of the drug pump.

21. The device of claim 20 wherein the sensor detects depletion of the flexible bladder and the feedback circuitry causes the drug pump to operate so as to fill the flexible bladder.

22. A drug-delivery device comprising:
(a) a patch adherable to a patient's skin; and
(b) integral with the patch and residing within an envelope defined by the patch,
(i) a lancet wirelessly actuable for insertion into a patient's skin in contact with the patch; and
(ii) at least one programmable drug pump comprising a reservoir, a cannula for conducting liquid from the reservoir to the lancet, and a mechanism for forcing liquid from the reservoir through the cannula and into a delivery vehicle.
